(19) United States
(12) Patent Application Publication
(10) Pub. No.: US 2008/0319432 A1
(43) Pub. Date: Dec. 25, 2008

(54) SURGICAL ILLUMINATION SYSTEM AND METHOD

(76) Inventors: Scott Ely, Cedar Park, TX (US); Jeffrey Brian Brown, Round Rock, TX (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(21) Appl. No.: 11/765,819

(22) Filed: Jun. 20, 2007

(51) Int. Cl. A61B 17/00

(52) U.S. Cl. 606/14

(57) ABSTRACT

The disclosure describes embodiments of systems and methods for surgical illumination, particularly for spinal procedures. Embodiments of the system comprise a surgical port that acts as an optical guide to guide light from an illumination conduit to an illuminator section. The illuminator section can be configured to direct light into a passage to illuminate a target area.

### TECHNICAL FIELD OF THE DISCLOSURE

Embodiments described in the disclosure relate to surgical illumination. Even more particularly, embodiments relate to illuminated surgical ports for use in spinal procedures.

### BACKGROUND OF THE DISCLOSURE

A number of maladies afflict the spine, causing severe pain, loss of mobility and decreased quality of life. Some examples of such disorders include degenerative disc disease, scoliosis, spinal deformities and other spinal conditions. Additionally, vertebral fractures and other trauma can cause spinal suffering.

Some conditions can be treated by surgical techniques such as spinal fusion. In spinal fusion, vertebrae are fused together by bone growth to immobilize the vertebrae and reduce pain. In spinal fusion procedures, a small interbody device of plastic, titanium or other biocompatible material is inserted between the vertebrae in place of the natural intervertebral disc.

Recently, minimally invasive surgery (“MIS”) has become more popular for spinal surgeries. In MIS, the surgeon makes one or more small incisions in the patient rather than a single large incision. In general, the surgeon attempts to make the incisions as small as possible to perform a procedure. It is believed that patients can recover in less time with less pain from MIS procedures than traditional procedures.

### SUMMARY OF THE DISCLOSURE

The disclosure provides embodiments of surgical systems and methods that provide illumination for procedures. In particular, various embodiments provide illumination for minimally invasive spinal surgeries such as discectomy procedures in the thoracic and/or thoracolumbar spine (T3-L5). One embodiment includes a surgical port that comprises a collar section and a passage section. The collar section can be configured to abut the patient during use, while the passage section extends into the patient. The surgical port can further include an optical coupler to couple to a light conduit, such as a fiber optic cable or light rod. The passage section comprises an outer surface and an inner surface. The inner surface at least partially defines a passage from a first end of the surgical port (e.g., outside of the patient) to a second end of the surgical port (e.g., proximate to a target area for a surgical procedure). The passage section further comprises an illuminator section comprising one or more features configured to direct light into the passage. The passage section is configured to act as an optical light guide to propagate light received from the light conduit to the illuminator section via internal reflection. Consequently, light from the light conduit can be directed into the passage and onto the target area.

Another embodiment can include a surgical illumination system comprising an illumination source, a light conduit coupled to an illumination source, a surgical assist mechanism and a surgical port coupled to the light conduit and the surgical assist mechanism. The surgical port can include a collar section configured to abut the patient during use while the passage section extends into the patient. The surgical port can further include an optical coupler to couple to a light conduit, such as a fiber optic cable or light rod. The passage section comprises an outer surface and an inner surface. The inner surface at least partially defines a passage from a first end of the surgical port (e.g., outside of the patient) to a second end of the surgical port (e.g., proximate to a target area). The passage section further comprises an illuminator section comprising one or more features configured to direct light onto features of the spine when the surgical port is in place in the patient. The passage section is configured to act as an optical light guide to propagate light received from the light conduit to the illuminator section via internal reflection. Consequently, light from the light conduit can be directed into the passage and onto the target area of the surgical procedure.

Another embodiment can include a method for minimally invasive spinal surgery comprising creating an incision in a patient, attaching a guide wire to the patient proximate to a target area for a spinal procedure, guiding a first dilator along the guide wire to widen a channel to the surgical site, guiding a set of sequentially larger dilators on the outside of previously inserted dilators until the channel is a desired size, inserting a surgical port into the incision on the outside of the largest dilator from the set of sequentially larger dilators, removing the dilators from the patient, and illuminating the target area with light passing from the surgical port. The surgical port can comprise a collar section comprising a first surface positioned to abut a patient's skin when the surgical port is in place, an optical coupler to couple to a light conduit and a passage section extending from the collar section. The passage section further comprises an outer surface, an inner surface at least partially defining a passage from a first end of the surgical port to a second end of the surgical port, and an illuminator section comprising one or more features configured to direct light into the passage. The passage section can be configured to act as an optical light guide to propagate light received from the light conduit to the illuminator section via internal reflection.

Embodiments of the systems and methods provide an advantage with respect to previous surgical ports for spinal surgery by eliminating or reducing reliance on overhead lighting or head mounted lights to illuminate a target area. This is advantageous as light from overhead and head mounted lights is often obscured by surgical tools or the members of the surgical team. Additionally, embodiments eliminate or reduce the need to insert additional illumination tools into the workspace, thereby reducing visual clutter and allowing more room for other tools.

### BRIEF DESCRIPTION OF THE FIGURES

A more complete understanding of the disclosure and the advantages of various embodiments may be acquired by referring to the following description, taken in conjunction with the accompanying drawings in which like reference numbers indicate like features and wherein:

FIG. 1 is a diagrammatic representation of an oblique view of one embodiment of an illumination system;

FIG. 2 is a diagrammatic representation illustrating light propagation from a surgical port;

FIGS. 3A and 3B are diagrammatic representations of an embodiment of a surgical port with a feature pattern for an illuminator section;

FIGS. 4A and 4B are diagrammatic representations of another embodiment of a surgical port with a feature pattern for an illuminator section;

FIGS. 5A and 5B are diagrammatic representations of another embodiment of a surgical port with a feature pattern for an illuminator section;

FIGS. 6A and 6B are diagrammatic representations of yet another embodiment of a surgical port with a feature pattern for an illuminator section;

FIG. 7 is a diagrammatic representation of a patient prepared to undergo spinal surgery;

FIG. 8 is a diagrammatic representation of locating a position for a guide wire;

FIG. 9 is a diagrammatic representation of dilating a channel;

FIG. 10 is a diagrammatic representation of determining a depth for selection of a surgical port;

FIG. 11 is a diagrammatic representation of a surgical port inserted in an incision;

FIG. 12 is a diagrammatic representation of surgical tools accessing a target area via a surgical port;

FIG. 13 is a flow chart for one embodiment of minimally invasive surgery;

FIG. 14 is a diagrammatic representation of another embodiment of a surgical port; and

FIG. 15 is a diagrammatic representation illustrating an embodiment of surgical port directing light to a passage and target area.

### DETAILED DESCRIPTION OF THE DISCLOSURE

Preferred embodiments are illustrated in the FIGURES, like numerals being used to refer to like and corresponding parts of the various drawings.

A surgical port provides access to a target area of a procedure. Various embodiments provide systems and methods for a surgical port in a spinal procedure using a port that provides illumination. In general, a port is a device that defines a passage from the exterior of a patient to a target area through which the target area can be viewed by a surgeon and accessed using surgical instruments. According to various embodiments, the port can be formed of a material or materials to act as an optical wave guide. Structures in the port can act as lenses or other optical structures to create an illuminator section that directs light into the passageway and/or onto the target area. This allows illumination of the target area without requiring the introduction of additional tools into the workspace. Eliminating ancillary instruments has the added benefit of further increasing the visibility of the target area by reducing clutter in the passageway.

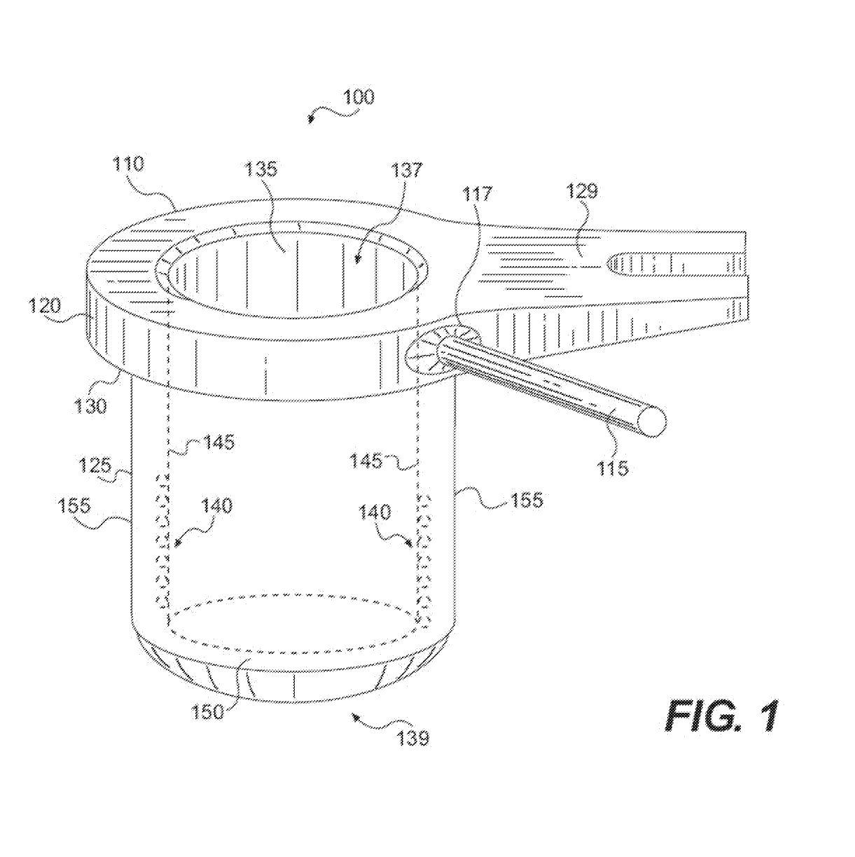

FIG. 1 is a diagrammatic representation of one embodiment of an illumination assembly 100 including a surgical port 110 coupled to a fiber optic cable 115. Fiber optic cable 115 can act as a waveguide to guide light from an illumination source (not shown) to surgical port 110 and can be formed of plastic or glass and can include plastic or glass cladding or other cladding. Fiber optic cable 115 can include a reflective sheath. Fiber optic cable 115 can be a graded-index, step-index, multiple-mode, single-mode, polarization maintaining, photonic crystal or other suitable optical fiber known in the art. Other light conduits, such as plastic or glass light rods or other light conduits known or developed in the art may also be used.

Surgical port 110 can include any optical coupler 117 known or developed in the art, to receive the optical fiber connector of fiber optic cable 115. Optical coupler 117 can be compatible with connectors including, but not limited to, a Wolf connector, Storz connector, Olympus connector, ACMI connector, Lucent connector, local connector, straight tip, subscriber connector, standard connector, Ferrule connector, Biconic connector, D4 connector, E2000 connector, Enterprise System Connection connector, Fiber Distributed Data Interface connector, Opti-Jack connector, Multi-Fibre Push On connector, Multi-Terminus connector, MTP connector, Mechanical Transfer Registered Jack connector, MU connector, Sub Miniature A connector, Sub Miniature C connector, Toshiba Link connector or other connector. Optical coupler 117 can include lenses that can be selected to match the acceptance angle of surgical port 110. Fiber optic cable can also be coupled to an illumination source, examples of which include, but are not limited to, xeon lights, arc lamps, incandescent bulbs, a lens end bulb, a line light, a halogen lamp, a light emitting diode (“LED”), an emitter from an LED, a neon light, a laser, a laser diode or other suitable light source.

Surgical port 110 can include a collar section 120 and a passage section 125. Collar section 120 and passage section 125 can be a single piece of material, preferably, a bio-compatible polycarbonate. In other embodiments, surgical port 110 can comprise multiple pieces that can be securely connected together. Collar section 120 can have a larger cross-sectional area than passage section 125 so that surface 130 of collar section 120 abuts the exterior of the patient around an opening when surgical port 110 is in place. Collar section 120 can include an extension with arms or other adaptations shaped so that surgical port 110 can be coupled to a snake arm or surgical assist mechanism (“SAM”) arm. For example, collar section 120 can include extension 129 adapted to couple to a snake arm. Collar section 120 can be a unitary piece or multiple pieces. As an example, extension 129 can couple to the remainder of collar section 120.

Passage section 125 extends from collar section 120 and defines a portion of passage 135 from a first end 137 to a second end 139 of surgical port 110. Through passage 135, a doctor can view and access a target area. Thus, passage section 125 can act as a cannula or lumen. While shown as generally cylindrical or tubular shape in FIG. 1, surgical port 110 can have any suitable form factor including square, oval or other shape. The end of passage section 125 at end 139 can be beveled, sloped or otherwise shaped to ease insertion of passage section 125 through tissue.

Passage section 125 can act as an optical waveguide to guide light received from fiber optic cable 115 to an illuminator section 140. In general, passage section 125 comprises a transparent or translucent light emitting material that is formed of an acrylic, polycarbonate, glass, epoxy, resins or other material. This is in contrast to traditional surgical ports that were preferably black to prevent glare. Preferably, collar section 120 is formed of the same material as passage section 125. Passage section 125 can include a single layer or multiple layers configured so that when passage section 125 is in the body, light in passage section 125 undergoes internal reflection, preferably total internal reflection (“TIR”), at both interior surface 145 and exterior surface 155 until the light reaches illuminator section 140. As is understood in the art, TIR occurs when light is incident on a surface at an angle of incidence that is greater than the critical angle. Preferably, no light escapes surgical port 110 except at the inner surface 145, end surface 150 and/or the outer surface 155 at illuminator section 140. In some embodiments, other than illuminator section 140, passage section 125 can act as a non-illuminator and not pass light to passage 135. A reflective or other coating may be disposed on the surfaces of surgical port 110 to assist TIR and prevent light leakage.

Illuminator section 140 can include any feature that allows light to be directed into passage 135 and/or onto the target area. Such features can include changes in material, surface coatings, geometries or other features that allow light to be extracted. For example, illuminator section 140 can include a layer of material or a surface coating that changes the index of refraction at illuminator section 140. As another example, illuminator section 135 can include geometric features on the inner surface of passage section 125 which cause light rays internal to passage section 125 to be incident on the features at angles that allow some or all of the light from the rays to refract into passage 135. For example, illuminator section 140 can include a variable pattern of features that allows light to be directed into passage 135 at illuminator section 140. Light may also pass from the end of passage section 125 through end surface 150 or to the area surrounding passage section 125 through outer surface 155. While shown as having a single illuminator section 140 that encircles passage 135 in FIG. 1, surgical port 110 may have multiple illuminator sections to direct light to different areas.

Geometric features can be produced using a printed pattern, an etched pattern, a machined pattern, a hot-stamped pattern or a molded pattern. In other embodiments, the pattern can be applied as a sheet or film to the surface of passage section 125 that either deforms or adheres to the surface of passage section 125 to define illuminator section 140. The light pattern produced by the geometric features can be controlled by varying the size, shape, opaqueness, translucence, color, index of refraction, diffraction grating or other properties of the features. The features can be configured to extract a larger percentage of available light closer to the target area than further up passage 135. This accounts for the fact that the light will likely have a greater intensity closer to optical fiber 115 than near the end of passage section 125 proximate to the target area. Because, a larger percentage of available light is extracted further away from optical fiber 115, where less light is available, the potentially nonuniform intensity profile in passage section 125 can be emitted in a more uniform pattern. According to one embodiment, the features can be configured to act as microlenses to direct light to a desired plane with a near-field profile. The microlenses can be shaped to provide diffuse or focused light. Other examples of light extracting features include prismatic surfaces, depressions or raised surfaces of various shapes. According to one embodiment, illuminator section 140 is approximately 0.75 to 1.00 inches high.

A surgical kit can include any number of surgical ports 110 having various dimensions, including diameters and lengths. The size or other parameter of a surgical port 110 can be indicated on the surgical port through alphanumeric characters, barcodes, color coding or other suitable mechanism. Each surgical port 110 can include features that assist in placement of surgical port 110. For example, surgical ports can include radiolucent markers or wires near end surface 150 so that the end of the surgical port 110 can be seen under medical imaging.

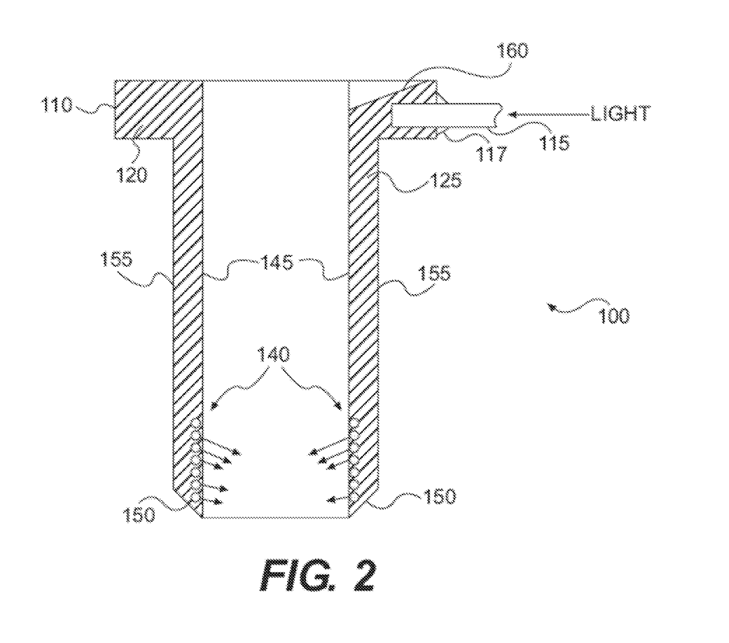

FIG. 2 is a diagrammatic representation of one embodiment of light entering and passing from illumination assembly 100. Optical fiber 115 can interface surgical port 110 through optical coupling 117 at collar section 120. In other embodiments, optical fiber 115 can interface with port 110 elsewhere, however, it is preferred that the interface be at a portion of surgical port 110 that will be outside of the patient during use. Surgical port 110 can include a surface 160, lens or other features to distribute light received from optical fiber 115 at suitable angles such that some amount—preferably a majority, and more preferably all of the light received from optical fiber 115—undergoes TIR when the light encounters inner surface 145 or outer surface 155. The light can propagate through surgical port 110 until it reaches illuminator section 140 where it encounters the features that cause the light to be directed into passage 135. Depending on configuration, light can also pass from end surface 150 and outside surface 155.

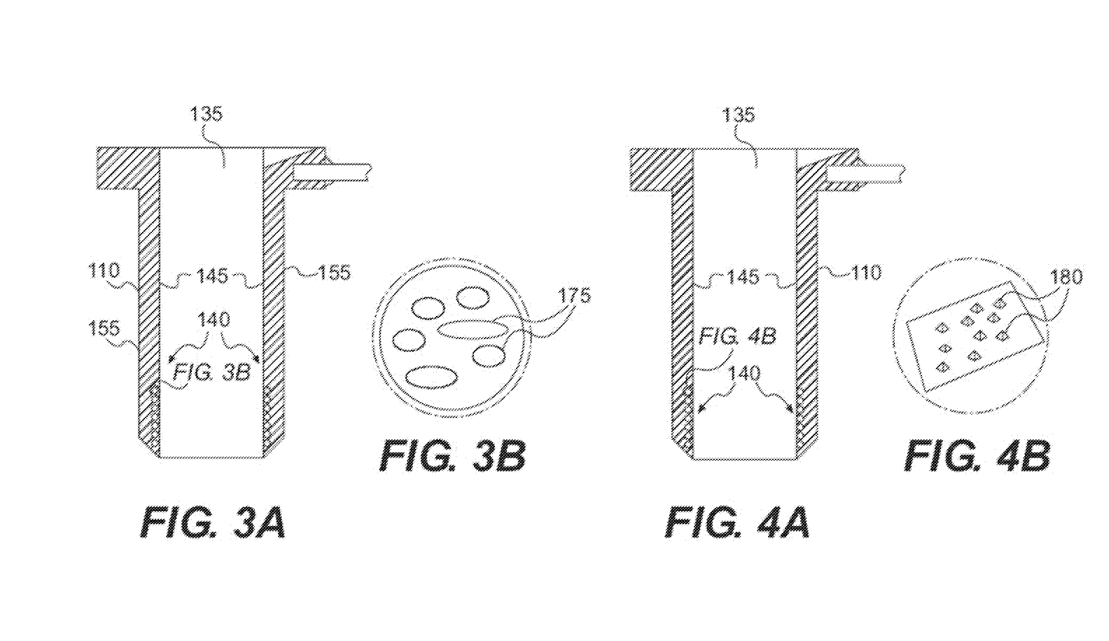

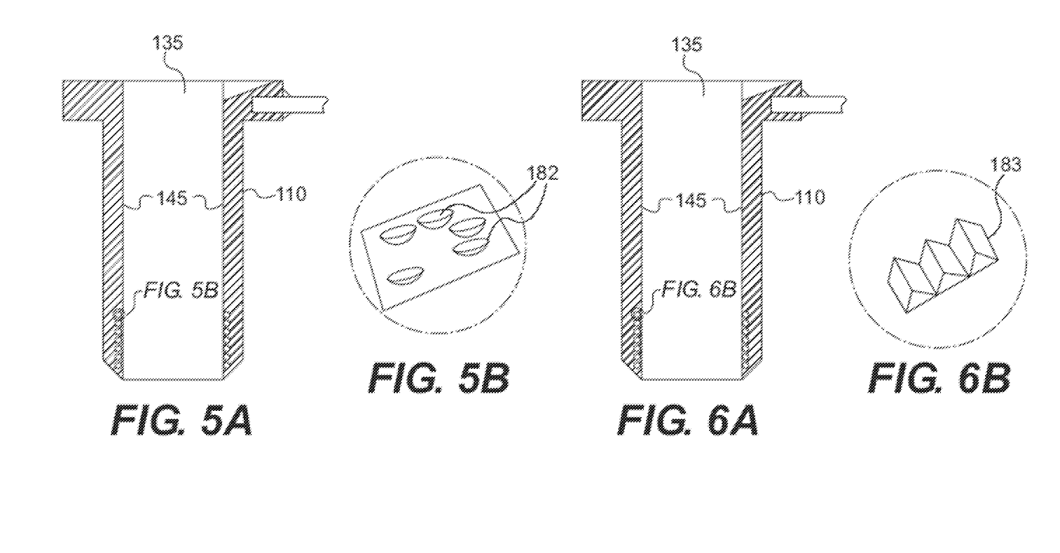

FIGS. 3A and 3B are diagrammatic representations of of a pattern of features 175 on the inner side of surface 145 for extracting light. Features 175 can be any change in the shape or geometry of the surface, surface coating or surface treatment of surgical port 110 that causes light to be extracted from surgical port 110 and be directed into passage 135. FIG. 3B illustrates a random pattern of features 175 that cause some portion of light incident on features 175 to be directed into passage 135. In another embodiment, surface 155 may include features that cause light to be reflected back to surface 145 at an angle such that the light will pass into passage 135 from surface 145 at illuminator section 140. Various embodiments of features can be used in random or nonrandom patterns. For example, FIGS. 4A and 4B are diagrammatic representations in which the features are prisms 180 formed on surface 145, FIG. 5A and 5B are diagrammatic representations in which the features are depressions 182 formed in surface 145 and FIGS. 6A and 6B are a diagrammatic representations of a “saw-tooth” prism pattern 183 disposed on surface 145. The features of FIGS. 3-6 may be located internal to surgical port 110 or may project from surface 145 into passage 135. While particular examples of features are shown in FIGS. 4-6, any feature can be used that extracts light from surgical port 110 including, but not limited to, features with curved sides, conical shapes, straight sides, parabolic or multi-parabolic based sides, multi-faceted sides or other features.

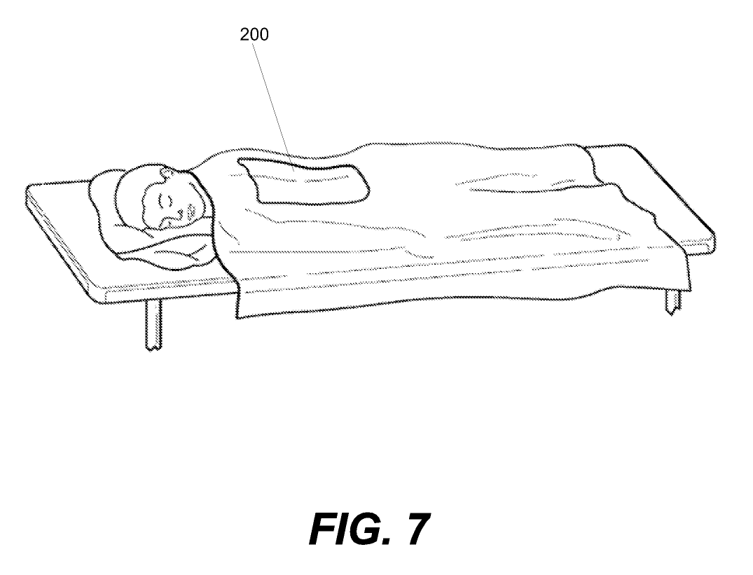

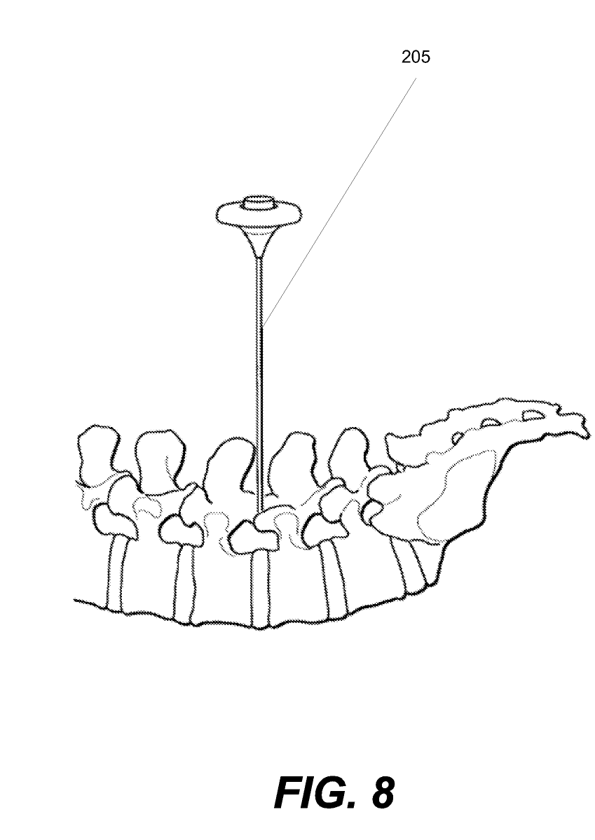

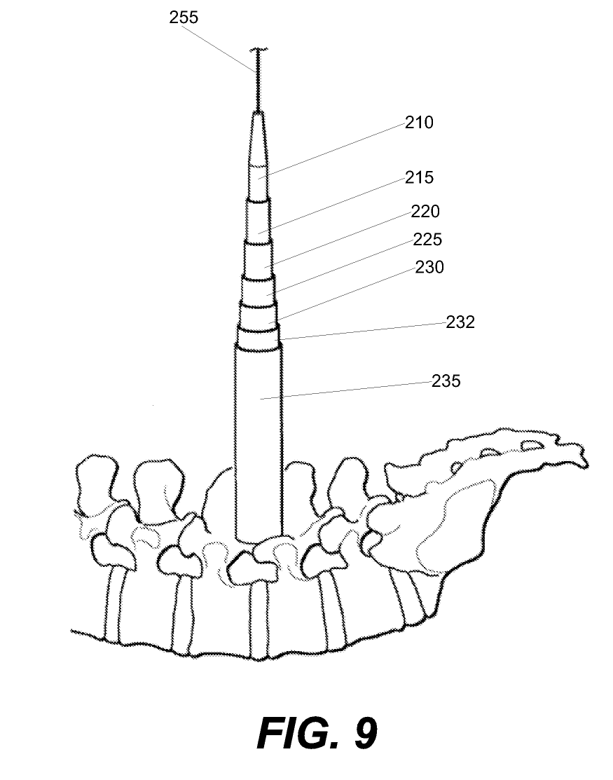

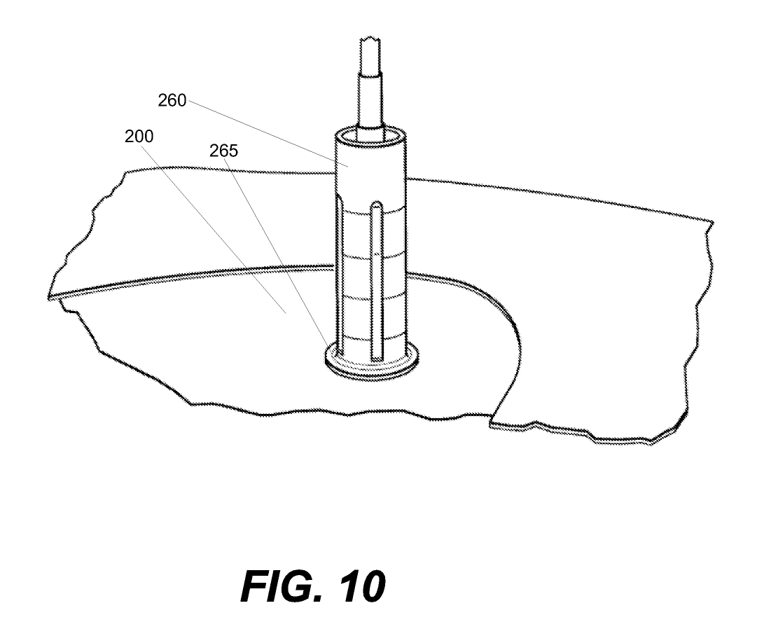

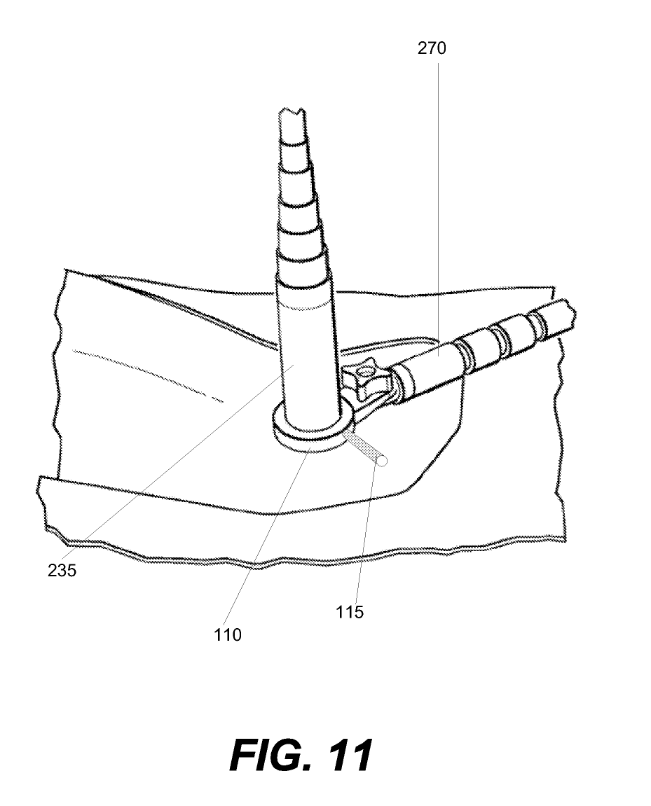

Surgical ports such as described in conjunction with FIG. 1 above can be used in MIS procedures, particularly posterior discectomy procedures in the thoracic and/or thoracolumbar spine (T3-L5). FIGS. 7-12 describe various steps in a posterior procedure. As shown in FIG. 7, a patient 200 can be positioned on a radiolucent table with clearance for a fluoroscopic C-arm for anterior, posterior, lateral and oblique images of pedicle and vertebral bodies. FIG. 8 illustrates that a surgeon can make a small incision in the patient's back and insert a targeting needle 205 to position a K-wire or other guide wire known or developed in the art (not shown) in the spine. The K-wire acts as a guide for subsequent components as described below. The surgeon can increase the size of the initial incision using a set of sequentially larger dilators. FIG. 9, for example, illustrates that a surgeon can sequentially slide dilatators 210, 215, 220, 225, 230, 232 and 235 over K-wire 255 to progressively expand the channel from the surface of the patient's body to the target area. As shown in FIG. 10, when the opening in the patient is the desired size, the surgeon can slide a depth gauge 260 over the largest dilator of FIG. 9. Flange 265 of depth gauge 260 rests against the skin of patient 200. The location of the top of the outermost dilator can be compared to markings on depth gauge 260 to determine the depth of the target area from the surface of the skin. This can be used to select the appropriate surgical port 110. The surgeon can slide the selected surgical port 110 over the largest dilator 235, as shown in FIG. 11. The angle of surgical port 110 can be adjusted by moving the dilators. Surgical port 110 can be attached to the snake arm 270 or other SAM and a fiber optic cable 115. The surgeon can remove the dilators when surgical port 110 is in place and activate the light source to illuminate the target area on the spine.

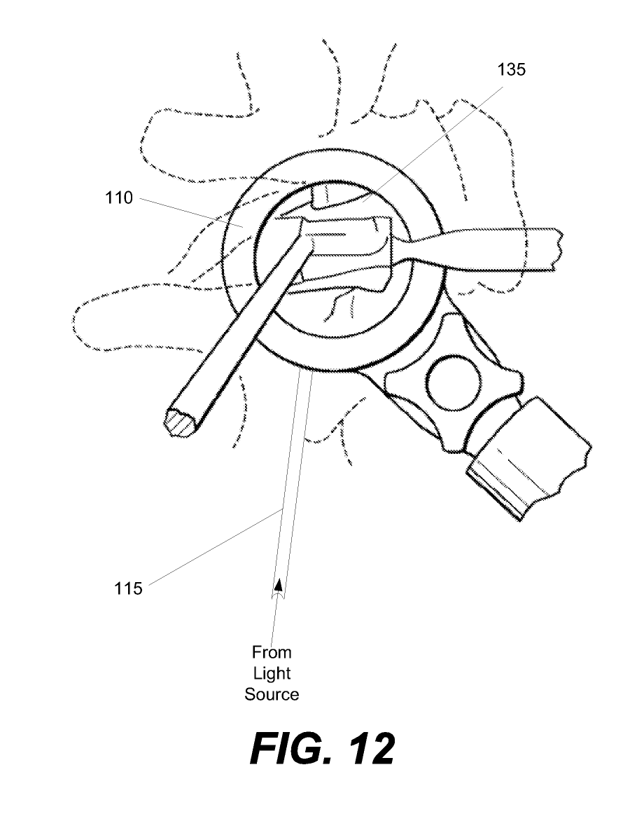

Surgical port 110 creates a passage through displaced soft tissue to the target area. As shown in FIG. 12, the surgeon can access the target area with tools through passage 135 provided by surgical port 110. For example, the surgeon can remove facets and portion of lamina, cut ligamentum flavum, free nerve root and dura from soft tissue, probe bony structures, retract nerve root and dura, remove blood and small tissue fragments, create annular windows, remove disc fragments, remove endplate cartilage, distract vertebrae, prepare vertebrae with a rasp, insert spinal devices such as interbody devices, plates, bone screws, rods and other devices and perform other spine related procedures through port 110. Examples of instruments that can be used through port 110 include, but are not limited to, osteotomes, curettes, probes, catheters, knives, rongeurs, distractors, rasps and other instruments. Because surgical port 110 provides illumination by distributing light received from fiber optic 115 to an illuminator section, the surgeon can see the target area without having to introduce additional illumination tools into passage 135, thereby reducing visual clutter. Additionally, the surgeon does not have to rely on overhead or head mounted lamps for illumination.

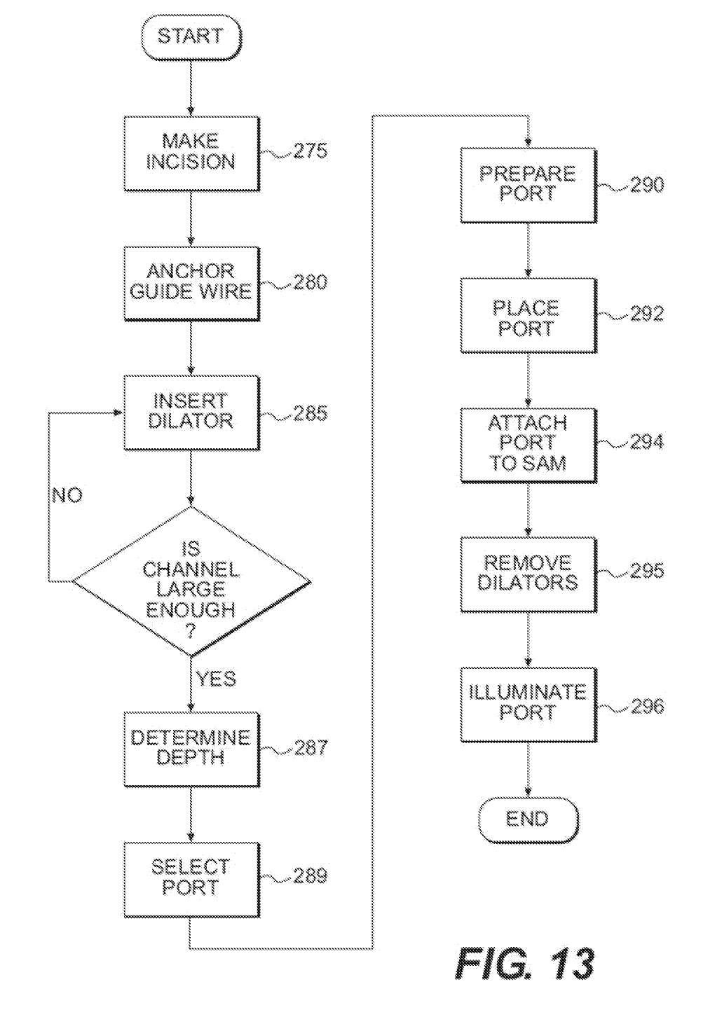

FIG. 13 is a flow chart for one embodiment of MIS. The surgeon can make an incision in the patent (step 275) and anchor a K-wire (or other guide wire) to a selected location in the spine (step 280). The surgeon can then slide a dilator along the K-wire to widen the channel to the target area and displace soft tissue (step 285). The surgeon can slide progressively larger dilators into the incision until the channel is wide enough. When the surgeon is satisfied with the size of the opening, the surgeon can measure the depth to the target area (step 287) and select a surgical port of sufficient depth (step 289). At step 290, the surgeon can prepare the surgical port by coupling it to an illumination source via an illumination conduit. The surgeon can place the port (step 292). This can include sliding the surgical port into the opening on the outside of the largest dilator until the surgical port is in place and adjusting the angle of the port by angling the dilators. Preferably, the surgical port is shaped so that a feature of the surgical port (such as surface 130, shown in FIG. 1), prevents the surgical port from being inserted too far into the patient. The surgeon can attach the surgical port to a SAM (step 294) to hold the port in the desired orientation. The surgeon can remove the dilators and optionally the guide wire if no longer needed to guide other components to the target area to create a clear passage to the target area (step 295). The surgical port can be illuminated at any point after it is optically coupled to the illumination source (shown at step 296). The steps of FIG. 13 can be repeated as needed or desired. Furthermore, the steps of FIG. 13 are provided by way of example and other methods can be used. Additionally, the steps of FIG. 13 can be performed in a different order. For example, it is not necessary to prepare the port before placing it. Additional steps can also be performed. For example, the surgeon may have to widen the incision using a scalpel or other cutting device.

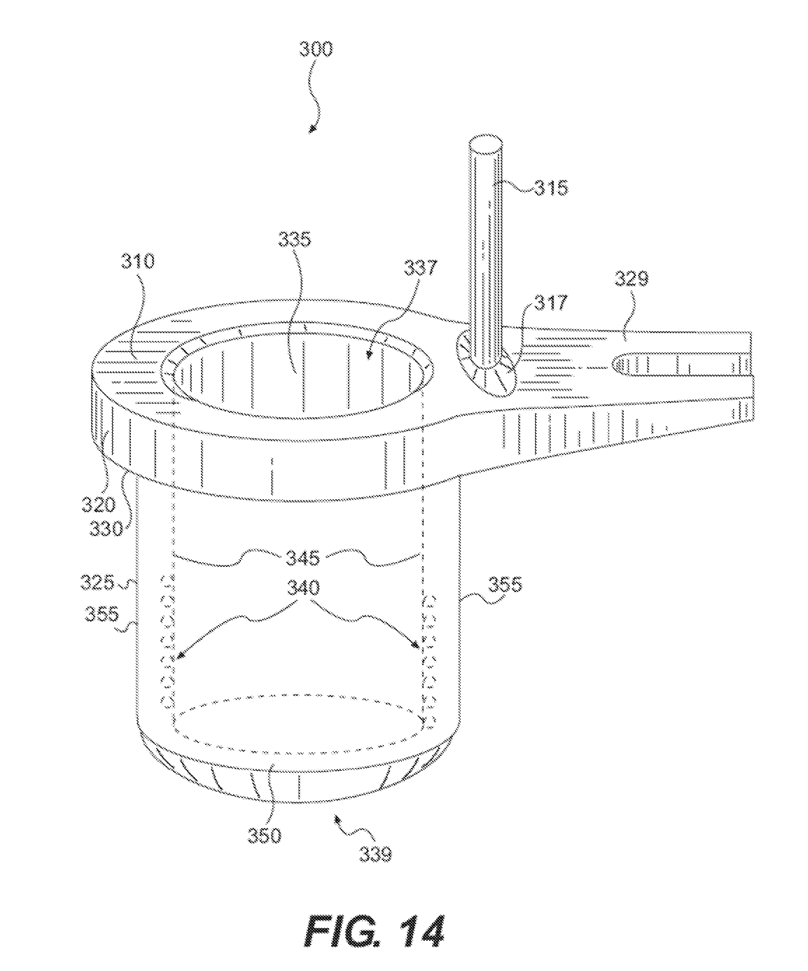

In the above examples, fiber optic cable 115 enters the side of surgical port 110 as shown, for example, in FIG. 1. FIG. 14, on the other hand, is a diagrammatic representation of another embodiment of an illumination assembly 300 including a surgical port 310 coupled to a fiber optic cable 315. Fiber optic cable 315 can act as a waveguide to guide light from an illumination source (not shown) to surgical port 310. Surgical port 310 can include any optical coupler 317 known or developed in the art to accept the termination connector of fiber optic cable 315. Optical coupler 317 can include lenses that can be selected to match the acceptance angle of surgical port 310. Optical fiber 315 can be a plastic fiber, glass fiber or other fiber. Surgical port 310 can couple to an illumination source in any other suitable manner. Examples of illumination sources include, but are not limited to, xeon lights, arc lamps, incandescent bulbs, a lens end bulb, a line light, a halogen lamp, a light emitting diode (“LED”), an emitter from an LED, a neon light, a laser, a laser diode or other suitable light source.

Surgical port 310 can include a collar section 320 and a passage section 325. Collar section 320 and passage section 325 can be a single piece of material, preferably, a bio-compatible polycarbonate. In other embodiments, surgical port 310 can comprise multiple pieces that can be securely connected together. Collar section 320 can have a larger cross-sectional area than passage section 325 so that surface 330 of collar section 320 abuts the exterior of the patient around an opening when surgical port 310 is in place. Collar section 320 can include a shaped section 329 that is shaped so that surgical port 310 can be coupled to a snake arm or other SAM. For example, collar section 320 can include arms adapted to couple to a snake arm. Collar section 320 can be a unitary piece or multiple pieces. For example, section 329 can attach to the remainder of collar section 320 via bonding, a screw, interference fit, rivet, bolt or other attachment mechanism.

Passage section 325 extends from collar section 320 to define a portion of passage 335 from a first end 337 to a second end 339 of surgical port 310. Through passage 335, a doctor can view and access a target area. Thus, passage section 325 can act as a cannula or lumen. While shown as generally cylindrical or tubular shape in FIG. 14, surgical port 310 can have any suitable form factor.

Passage section 325 can act as an optical waveguide to guide light received from fiber optic cable 315 (or other light conduit) to an illuminator section 340. In general, passage section 325 comprises a transparent or translucent light emitting material that is formed of an acrylic, polycarbonate, glass, epoxy, resins or other material. Passage section 325 can include a single layer or multiple layers configured so that when passage section 325 is in the body, light in passage section 325 undergoes internal reflection, preferably TIR at both interior surface 345 and exterior surface 355 of passage section 325 until the light reaches illuminator section 340. Preferably, no light escapes surgical port 310 except at the inner surface 345, end surface 350 and/or the outer surface 355 of passage section 325 at illuminator section 340. A reflective or other coating may be disposed on the surfaces of surgical port 310 to assist TIR and prevent light leakage.

Illuminator section 340 can include any feature that allows light to be extracted from surgical port 110 and directed into passage 335 and/or onto the target area. Such features can include changes in material, surface coatings, geometries or other features that allow light to be extracted. For example, illuminator section 340 can include a layer of material or a surface coating that changes the index of refraction at illuminator section 340. As another example, illuminator section 335 can include geometric features on the inner surface of passage section 325 which cause light rays internal to passage section 325 to be incident on the features at angles that allow some or all of the light from the rays to refract into passage 335. For example, illuminator section 340 can include a variable pattern of features that allows light to pass into passage 335 at illuminator section 340. Light may also pass from the end of passage section 325 through end surface 350 or to the area surrounding passage section 325 through outer surface 355. While shown as having a single illuminator section 340 that encircles passage 335 in FIG. 14, surgical port 310 may have multiple illuminator sections to provide light in different areas.

Geometric features can be produced using a printed pattern, an etched pattern, a machined pattern, a hot-stamped pattern or a molded pattern. In other embodiments, the pattern can be applied as a sheet or film to the surface of passage section 325 that either deforms or adheres to the surface of passage section 325 to define illuminator section 340. The light pattern produced by the geometric features can be controlled by varying the size, shape, opaqueness, translucence, color, index of refraction, diffraction grating or other properties of the features. The features can be configured to extract a larger percentage of available light closer to the target area than further up passage 335. This accounts for the fact that the light will likely have a greater intensity closer to optical fiber 315 than the end of passage section 325 proximate to the target area. Because, a larger percentage of available light is extracted further away from optical fiber 315, where less light is available, the potentially nonuniform intensity profile in passage section 325 can be emitted in a more uniform pattern. According to one embodiment, the features can be configured to act as microlenses to direct light to a desired plane with a near-field profile. The microlenses can be shaped to provide diffuse or focused light. Other examples of light extracting features include prismatic surfaces, depressions or raised surfaces of various shapes. According to one embodiment, illuminator section 340 is approximately 0.75 to 1.00 inches high.

A surgical kit can include any number of surgical ports 310 having various dimensions, including diameters and lengths. The size or other parameter of a surgical port 310 can be indicated on the surgical port through alphanumeric characters, barcodes, color coding or other suitable mechanism. Each surgical port 310 can include features that assist in placement of surgical port 310. For example, surgical ports can include radiolucent markers or wires near end surface 355 so that the end of the surgical port 310 can be seen under medical imaging.

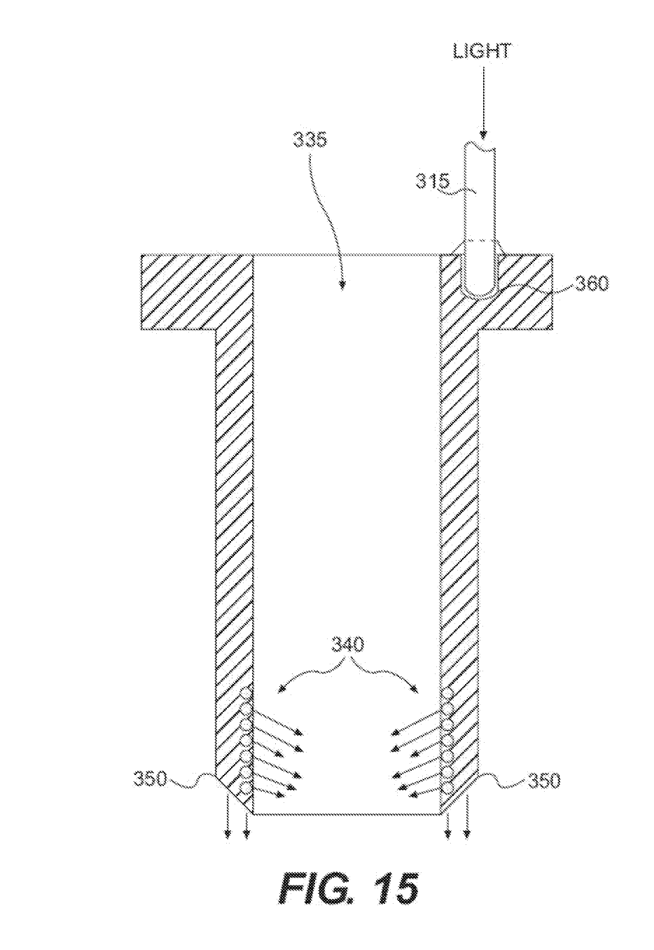

FIG. 15 is a diagrammatic representation of one embodiment of light traveling through illumination assembly 300. Optical fiber 315 can interface surgical port 310 through any suitable optical coupling. Surgical port 310 can include a notch, groove or other suitable feature 360 to engage optical fiber 315. Feature 360 can be selected to optimize light coupling. The light can propagate through surgical port 310 until it reaches illuminator section 340 where it encounters the features that cause the light to be directed into passage 335. Additionally, in this embodiment, light can pass from end surface 350.

Embodiments provide a surgical port with a passage section that acts as a waveguide for light received from a light conduit (e.g., an optical fiber). The surgical port may be used in spinal surgery, such as discectomies or microdiscectomies. The port can provide access to and illumination of a target area for implantation of interbody devices, bone anchors, fixation devices, plates, cables, artificial discs, and other devices. In the above embodiments, surgical port 110 receives light from a single light conduit. Other embodiments can be coupled to multiple light conduits. The multiple light conduits can all provide the same type of light or various colors or wavelengths of light.

While disclosure described particular embodiments, it should be understood that the embodiments are illustrative and that the scope of the claims is not limited to these embodiments. Many variations, modifications, additions and improvements to the embodiments described above are possible. It is contemplated that these variations, modifications, additions and improvements fall within the scope of the claims.

What is claimed is:

1. A surgical port comprising:
a collar section comprising a first surface positioned to abut a patient's skin when the surgical port is in use;
an optical coupler to couple to a light conduit;
a passage section extending from the collar section, the passage section further comprising:
an outer surface;
an inner surface at least partially defining a passage from a first end of the surgical port to a second end of the surgical port;
an illuminator section comprising one or more features configured to direct light from the surgical port into the passage;
wherein the passage section is configured to act as an optical light guide to propagate light received from the light conduit to the illuminator section via internal reflection.

2. The illumination system of claim 1, wherein the illuminator section is configured to extract more light in a first portion than a second portion, wherein the first portion is further from the light conduit than the second portion.

3. The illumination system of claim 1, wherein the one or more features comprise geometric features.

4. The illumination system of claim 1, wherein the one or more features comprise a set of microlenses.

5. The illumination system of claim 4, wherein the set of microlenses are shaped to provide focused light.

6. The illumination system of claim 4, wherein the set of microlenses are shaped to provide diffused light.

7. The illumination system of claim 1, wherein the one or more features are configured to provide a generally uniform intensity profile.

8. The illumination system of claim 1, wherein the passage section comprises a substantially tubular body.

9. The illumination system of claim 1, wherein the illuminator section comprises an end surface.

10. The illumination system of claim 9, wherein the end surface is a portion of the illuminator section.

11. The illumination system of claim 1, wherein the one or more features are further configured to direct light out of the surgical port from the outer surface of the passage section.

12. The illumination system of claim 1, wherein the collar section is adapted for attachment to a surgical assist mechanism.

13. The illumination system of claim 1, wherein the illuminator section is disposed from a portion of the passage section proximate to the second end to less than one inch from the second end.

14. A spinal surgical system comprising:
an illumination source;
a light conduit coupled to the illumination source;
a surgical port comprising:
a collar section comprising a first surface positioned to abut a patient's skin when the surgical port is in use;
an optical coupler to couple to the light conduit;
a passage section extending from the collar section in a direction to extend into the patient's body during use, the passage section further comprising:
an outer surface;
an inner surface at least partially defining a passage from a first end of the surgical port to a second end of the surgical port; and
an illuminator section comprising one or more features configured to direct light onto one or more spinal features when the surgical port is inserted in the patient;
wherein the passage section is configured to act as an optical light guide to propagate light received from the light conduit to the illuminator section via internal reflection;
a surgical assist mechanism coupled to the collar section of the surgical port.

15. The spinal surgical system of claim 14, wherein the illuminator section is configured to extract more light in a first portion than a second portion, wherein the first portion is further from the light conduit than the second portion.

16. The spinal surgical system of claim 14, wherein the one or more features comprise geometric features.

17. The spinal surgical system of claim 14, wherein the one or more features comprise a set of microlenses.

18. The spinal surgical system of claim 17, wherein the set of microlenses are shaped to provide focused light.

19. The spinal surgical system of claim 17, wherein the set of microlenses are shaped to provide diffuse light.

20. The spinal surgical system of claim 14, wherein the one or more features are configured to provide a generally uniform intensity profile.

21. The spinal surgical system of claim 14, wherein the passage section comprises a substantially tubular body.

22. The spinal surgical system of claim 14, wherein the illuminator section comprises an end surface.

23. The spinal surgical system of claim 14, wherein the end surface is a portion of the illuminator section.

24. The spinal surgical system of claim 14, wherein the one or more features are further configured to direct light out of the surgical port from the outer surface of the passage section.

25. The spinal surgical system of claim 14, wherein the collar section is adapted for attachment to a surgical assist mechanism.

26. The spinal surgical system of claim 14, wherein the illuminator section is disposed from a portion of the passage section proximate to the second end to less than one inch from the second end.

27. A method for minimally invasive spinal surgery comprising:
creating an incision in a patient;
attaching a guide wire to the patient proximate to a target area for a spinal procedure;
guiding a first dilator along the guide wire to widen a channel to the target area;
guiding a set of sequentially larger dilators on the outside of previously inserted dilators until the channel is a desired size;
inserting a surgical port into the incision on the outside of the largest dilator from the set of sequentially larger dilators, wherein the surgical port comprises:
a collar section comprising a first surface positioned to abut a patient's skin when the surgical port is in place;
an optical coupler to couple to a light conduit;
a passage section extending from the collar section, the passage section further comprising:
an outer surface;
an inner surface at least partially defining a passage from a first end of the surgical port to a second end of the surgical port;
an illuminator section comprising one or more features configured to direct light from the surgical port into the passage;
wherein the passage section is configured to act as an optical light guide to propagate light received from the light conduit to the illuminator section via internal reflection;
removing the first dilator and set of sequentially larger dilators from the patient; and
illuminating the target area with light from the surgical port.

28. The method of claim 27, further comprising determining a depth and selecting the surgical port from a set of surgical ports of various sizes based on the determined depth.

29. The method of claim 27, further comprising coupling the surgical port to the illumination source via the light conduit.

30. The method of claim 27, further comprising coupling the surgical port to a surgical assist mechanism.

31. The method of claim 30, further comprising adjusting an angle of the surgical port by adjusting the angle of at least one dilator prior to removing the first dilator and set of sequentially larger dilators from the incision.
